(19) United States
(12) Patent Application Publication
(10) Pub. No.: US 2020/0230334 A1
(43) Pub. Date: Jul. 23, 2020

(54) Single-Dose Powder-Medication Inhaler

(71) Applicant: Perlen Packaging AG, Perlen (CH)

(72) Inventors: Klaus-Dieter Beller, Kenzingen (DE); Bernd Kaiser, Endingen (DE)

(21) Appl. No.: 16/632,339

(22) PCT Filed: Jun. 22, 2018
(86) PCT No.: PCT/EP2018/000313

§ 371 Date: Jan. 18, 2020

(30) Foreign Application Priority Data
Jul. 18, 2017 (DE) 10 2017 006 763.5

(51) Int. Cl. A61M 15/08; A61M 15/00

(52) U.S. Cl. CPC A61M 15/08; A61M 15/0021; A61M 15/0043; A61M 2205/0205; A61M 2205/27; A61M 2206/14; A61M 2202/064

(57) ABSTRACT

The present invention provides a single-dose powder-medication inhaler (10) for nasal application, comprising a flat inhaler housing consisting of two flat housing parts (1, 2) connected to one another along a connecting plane (E). Formed in the two flat housing parts are: a medication chamber (11) having at least one powder receiving hollow (11′); an air inlet opening (14); and an air channel delimited by walls (7), the air channel extending from the air inlet opening (14), via the medication chamber (11) to a discharge opening (12). According to the invention the air inlet opening (14) does not lie on a side facing away from the discharge opening (12), whereby the air channel provides a direction change of an air flow through the air inlet opening (14) relative to the discharge opening (12). The outlet section (13) of the air channel between the medication chamber (11) and the discharge opening (12) has more than one bend (6). Furthermore a nose piece (4), designed for inserting in only one nostril or in both nostrils, is formed on the discharge opening (12).

The invention relates to a single-dose powder inhaler according to the preamble of claim 1.

The powder inhalers (“inhaler”) customarily available on the market are often designed for repeat use and are therefore complicated and expensive to produce. Use as a disposable article is often unsuitable. However, repeat use causes some significant hygiene problems since, after use of the inhaler, the patient cleans it incorrectly, rarely or not at all. A further problem is that inhaler caps are mislaid. From a hygiene point of view, it is also unfavorable if the patient uses the inhaler wrongly, for instance by breathing out into the inhaler: in known inhalers, patients do not always breathe out in the predefined exhalation direction, but instead through the inhaler, such that the inhaler tends to clog up on account of the humidity of the respiratory air upon repeat use, since the pulverulent medicament forms lumps and remains stuck in the air channel in the inhaler. These deposits and agglutinations, which may also be contaminated with pathological germs, can result in dosing inaccuracies and further drawbacks, particularly for nasal application: there, nasal germs should be taken into consideration, e.g. when sinusitis proves resistant to therapy. It is possible that the persons affected reinfect themselves (nasally, oropharyngeally) via the inhaler.

WO2007/042822 describes a single-dose powder inhaler of simple design having a first housing part and a second housing part which are made of metal and which, when joined together, form an inhaler housing with an outlet. The first housing part comprises a medicament chamber which contains a single dose of a pulverulent medicament and which is closed by a film secured to the first housing part. This film extends out of the inhaler housing, such that it can be gripped and pulled off for use by a user, with the result that the pulverulent medicament is released from the medicament chamber. The second housing part has a collection well and/or a dispersion chamber through which an air channel formed in the inhaler housing leads to the outlet. After the medicament chamber has been opened, an air flow is generated, upon inhalation at the outlet, through the gap between the housing parts that remains after the film has been pulled off, which air flow entrains the pulverulent medicament, released from the medicament chamber, out of the collection well or the dispersion chamber. However, said inhaler is not provided and designed for nasal application.

DE 10 2014 017 409 A1 discloses a single-dose powder inhaler likewise of simple construction, composed of an inhaler housing having a housing part in which at least one medicament chamber is formed for receiving a pulverulent medicament. The inhaler housing has an outlet opening, and an outlet channel extending from the medicament chamber to the outlet opening. An air inlet opening, on a side of the medicament chamber facing away from the outlet opening, and an inlet channel, extending from the air inlet opening to the medicament chamber, are likewise formed in the housing part, which is covered with a cover element after the medicament chamber has been filled with a powder dose. Said document further discloses an embodiment in which, for nasal application, the outlet opening of the inhaler is connected to a tapering nose nozzle. However, the inhaler itself is intended primarily for oral application and is designed accordingly, especially also as regards the deagglomeration of the powder contained in it.

GB 2 460 281 A discloses an inhaler composed of at least three layers laminated on each other with a thickness of less than 3 mm. The middle layer has cutouts in order to delimit, together with a cover layer and a base layer, an air channel, at least one medicament chamber and an air outlet region. The cover layer can have an air inlet opening, which comes to lie over an inlet region of the air channel of the middle layer, and ventilation openings, which come to lie over each medicament chamber. Air inlets can also be present between base and cover layer through recesses in the middle layer, on the side directed away from the outlet or on the adjacent side, from which straight or angled air channels open into the medicament chamber. Moreover, an outlet portion of the air channel can be wound between the medicament chamber and the outlet opening.

A powder inhaler composed of three housing parts is known from WO 2013/036881 A2. Two housing parts enclose the air inlet region and the medicament chamber; the powder/air outlet region toward the mouthpiece is a separate component. There, an annular chamber for the powder is formed in one housing part, while air inlets, which open into the annular chamber after joining together, and an outlet grating are formed in the other housing part. The separate outlet mouthpiece is secured over the outlet grating. The air inlets are oriented such that air is directed to the bottom of the annular chamber at a non-tangential angle, wherein in particular two air inlets are proposed which open into the annular chamber from opposite directions. To prevent powder being lost from an air inlet opening when the inhaler is inclined, the air inlet channel can have an additional winding.

A therapeutic distinction is made between nasal and oral application routes, especially as regards the deposition targets for the pulverulent medicament. Thus, nasal application targets the mucosa of the nasopharynx, while oral inhalation targets the mucosa of the oropharyngeal space, and both target deposition of the aerosol particles in the lungs after inhalation through the nose or mouth. Besides a locally limited therapeutic effect, it is therein also possible to achieve a systemic therapeutic effect. The location at which the inhaled powder particles are deposited depends among other things on the particle size.

Proceeding from this prior art, the object of the present invention is to make available a single-dose powder inhaler which is particularly compact and is optimized in particular for nasal application.

This object is achieved by a single-dose powder inhaler having the features of claim 1. Developments are specified in the dependent claims.

The single-dose powder inhaler according to the invention is also provided particularly advantageously for the nasal application of pulverulent medicaments. Nasal application is intended to permit deposition especially on the nasopharyngeal mucosa. Particle sizes in the region of 3 to 6 μm are suitable here; upward or downward deviations are permissible.

The single-dose powder inhaler according to the invention therefore has a flat inhaler housing consisting of only two planar housing parts connected to each other along a connecting plane. An air channel delimited by a wall on both sides is formed in the housing parts, which air channel widens around a shaped powder-receiving hollow for a dose of pulverulent medicament in order to form a medicament chamber. The air channel extends here between an air inlet opening and an outlet opening which are shaped or formed in the housing parts, wherein the air inlet opening is not arranged on a side of the inhaler facing away from the outlet opening, i.e. imaginary axes through air inlet opening and outlet opening are neither in alignment nor parallel. The course of the air channel provides a change of direction of an airflow through the air inlet opening relative to the outlet opening and has an outlet portion between the medicament chamber and the outlet opening, which outlet portion has more than one winding. That is to say, in a single-dose powder inhaler according to the invention, the air inlet opening and the outlet opening are arranged relative to each other in such a way that an air inlet flow direction, which is defined by the arrangement of the air inlet opening, differs from an air outlet flow direction, which is defined by the arrangement of the outlet opening, such that the air flowing out does not leave the inhaler in a straight-ahead direction with respect to the inflowing air, i.e. the air inlet opening and outlet opening are not arranged on sides of the inhaler that face away from each other. This arrangement of air inlet opening and outlet opening deviating from the “straight-ahead direction” is important in nasal application, since here the inhaler, in contrast to oral application, has to be held such that the outlet opening points substantially perpendicularly upward, into the nostrils, and therefore the deviating arrangement of the air inlet opening prevents a situation where the latter faces downward during use. Advantageously, during use, no powder can therefore be lost through the air inlet opening, which is something that could happen in a conventional powder inhaler in which the air inlet opening is arranged on a side directed away from the outlet opening, if, during the nasal application with the outlet opening at the nostrils, the air inlet opening points vertically downward.

The single-dose powder inhaler according to the invention for nasal application has a nosepiece which is integrally formed at the outlet opening and which is designed to be received in one nostril or in both nostrils. Designing the nosepiece to be received in only one nostril makes sense particularly in combination with two powder-receiving hollows for two partial doses. In order to control the flow speed, a portion of the inlet channel adjoining the air inlet opening can taper from the air inlet opening in the direction of the medicament chamber. The portion of the outlet channel opening into the outlet opening tapers in the direction of the outlet opening to the cross section of the nosepiece.

It is not ruled out that a single-dose powder inhaler according to the invention can be used for oral inhalation, for example if deposition of the powder in the oral or pharyngeal mucosa is desired. If appropriate, a mouthpiece can additionally be provided for this purpose, which mouthpiece can be fitted onto the nosepiece.

During the use of the inhaler, the user breathes in at the outlet opening to generate an air flow through the inhaler, such that the powder particles provided in a predetermined medicament dose in the powder-receiving hollow are entrained with the air flow. To ensure that the powder particles are distributed uniformly and readily in the air flow during the travel between the medicament chamber and the outlet opening, before they are inhaled by the user, an outlet portion of the air channel lying between the medicament chamber and the outlet opening has more than one winding, such that the air flow guided through the outlet channel portion during use performs several changes of direction. The single-dose powder inhaler according to the invention resigns from additional deagglomerating structures, since further comminution of the powder particles entrained in the airflow and thus subjected to a swirling motion is unnecessary and also undesirable.

Advantageously, the powder inhaler according to the invention is composed only of two housing parts, in which all the relevant portions are formed. The powder inhaler can therefore be produced very cost-effectively and is thus suitable for administering a dose of powder in a single use, with the associated advantages for the user as regards hygiene.

The air inlet opening and the outlet opening are arranged relative to each other in such a way that, between the air inlet flow direction defined by the arrangement of the air inlet opening and the air outlet flow direction defined by the arrangement of the outlet opening, an angle is formed in a range of 90° (perpendicular to the straight-ahead direction) and 180° (an about-turn as it were). An angle of 180° between the directions of flow signifies that the directions of air flow at the inlet and at the outlet are counter to each other, consequently present at the same side of the powder inhaler. Thus, the air inlet opening and the outlet opening are either arranged next to each other at the same side or are offset from each other by 90° on adjacent sides or at an angle lying therebetween, but not on sides facing away. Angles of less than 90° are also conceivable for the deviation from the straight-ahead direction. However, in order to effectively prevent the unwanted escape of powder from the air inlet opening, a deviation of at least 90° from the straight-ahead direction is expedient in order to avoid an air inlet opening facing downward during use.

The arrangement of the air inlet opening and of the outlet opening on the single-dose powder inhaler according to the invention can also vary in the sense that the air inlet opening and the outlet opening are arranged both perpendicular or both parallel to the connecting plane of the housing parts. Provision can also be made that one of the two openings, namely air inlet opening and outlet opening, is arranged in a plane parallel to the connecting plane, and the other opening is arranged perpendicular thereto.

The variants in which air inlet opening and outlet opening are arranged next to each other are particularly preferred, since almost no operating error can then arise; powder cannot in principle fall out, regardless of how the inhaler is held during use.

As regards the configuration of the outlet portion of the air channel between the medicament chamber and the outlet opening of a single-dose powder inhaler according to the invention, a meandering channel with at least three curves as windings can be provided, wherein 90° or 180° curves or a combination of both may be preferred. In the meandering channel, the air flow experiences several changes of direction. Alternatively to a meandering channel, the outlet portion can also be designed as a spiral-shaped channel with at least two windings. In the spiral-shaped channel, the air flow experiences a continuous change of direction. The number and the degree of curvature of the windings in the meandering channel and in the spiral-shaped channel also depend on the arrangement of the outlet opening with respect to the position of the medicament chamber. A spiral-shaped channel has an ideal cyclone effect, i.e. a particularly good air turbulence property.

To provide that the pulverulent medicament remains in the powder-receiving hollow until the time of use and is protected there from environmental effects during storage or transport of the single-dose powder inhaler, the powder-receiving hollow in a preferred embodiment is closed after it has been filled with the powder dose. For this purpose, a closure element is provided which can be easily removed by the user before use. As a detachable closure for the powder-receiving hollow, the single-dose powder inhaler can have a tear-off film element. A closure tab of the film element closes the one powder-receiving hollow in the medicament chamber, and a tear-off tab of the film element extends from the closure tab out of the medicament chamber and out of the inhaler, which tear-off tab can be gripped by the user such that, by pulling on the tear-off tab, he can tear the closure tab from the powder-receiving hollow and thus make the inhaler ready for use.

In one embodiment, provision can be made that the tear-off tab of the film element extends out of the air inlet opening through an inlet portion of the air channel between the air inlet opening and the medicament chamber. Therein, the inlet portion runs in a straight line between the medicament chamber and the inlet opening.

In an alternative embodiment to this, the inhaler can have an additional tab channel which extends in a straight line from the medicament chamber as far as a tab opening, such that the tear-off tab of the film element can here extend through the tab channel and out of the tab opening. Like the air inlet opening, the tab opening is arranged with respect to the outlet opening in such a way that, during the use of the inhaler with the outlet opening at the nose of the user after removal of the film element, no powder from the medicament chamber can trickle through the tab channel and out of the tab opening. In fact, after removal of the film element, the tab opening also acts as an additional air inlet opening, as a result of which, during use, two air flows can enter the medicament chamber, and the swirling and entrainment of the powder particles from the powder-receiving hollow can thus be still further improved.

In order to ensure, even after removal of the closure element and when handling a single-dose powder inhaler according to the invention, that no powder can fall out of the air inlet opening, or out of the tab opening if present, the inlet portion of the air channel, or the tab channel, can have a powder-retaining structure which narrows the channel cross section. Such a powder-retaining structure can preferably be formed by a barrier that extends into the air channel (or tab channel) transversely with respect to the direction of flow. Particularly preferably, the powder-retaining structure can be formed by several barriers or barrier portions alternately arranged opposite and/or next to each other, such that the path through the powder-retaining structure is in chicane-style or labyrinth-style.

Moreover, the inlet portion of the air channel, like the outlet portion, can also have more than one winding, such that the air flow that can be guided through the inlet channel portion also performs several changes of direction, with the result that a turbulent flow of air enters the medicament chamber, in order to swirl up and entrain the powder lying there in the powder-receiving hollow. Specifically in this embodiment of the powder inhaler with a sinuous inlet portion, it is expedient to provide a tab channel for tearing off the film element, since a sinuous inlet channel does not permit or does not readily permit the pulling through of the film element.

The single-dose powder inhaler according to the invention is provided for single use. However, this does not mean that the powder dose has to be present only in a single powder-receiving hollow. Instead, particularly in the case of nasal use for a therapeutic administration, an application of two partial doses can be provided individually in each of the nostrils. Thus, in a powder inhaler according to the invention, it is also possible to provide two powder-receiving hollows in the housing parts, each containing a (partial) dose of pulverulent medicament in the medicament chamber.

Here too, each powder-receiving hollow can be closed by the closure tab of a respective tear-off film element such that, during use, one powder-receiving hollow per nostril can be opened in succession and the powder can be released. Moreover, in an inhaler according to the invention, it is possible to provide two (or even more) powder-receiving hollows for the case in which different components of a pulverulent medicament are to be mixed together only at the time of use. In this case, the different components of the medicament dose can be stored in corresponding powder-receiving hollows, which can here also be closed by a common film element if the components are intended to be inhaled together.

The two housing parts of a single-dose powder inhaler according to the invention can be formed from plate elements in which all the structures are embodied in such a way that they rise from the flat plate base. The housing parts composed of plate elements can then be connected to each other in a planar manner in the connecting plane outside the shaped regions (after the powder-receiving hollow has been filled). Preferably, in order to achieve a more stable housing and better handling by a user, the two housing parts can however be formed from shell elements with circumferential edges, wherein the housing parts are then connected to each other in the connecting plane at least at these edges, but preferably also at walls delimiting the channel.

After the powder-receiving hollow has been closed with a film element, wherein the closure tab is sealed or adhesively bonded to the medicament chamber and the film portion leading to the tear-off tab is folded or turned back, the housing parts can be connected by sealing, adhesive bonding or also by welding (e.g. ultrasonic welding).

It is advantageously cost-effective that the housing parts of the powder inhaler can each be injection-molded plastic parts or can each be shaped, preferably thermoformed, from a pharmaceutically acceptable, preferably single-type plastic film. The plastic used, for at least one of the housing parts, can be a transparent plastic, such that the powder present in the medicament chamber and the administration of said powder can be checked by sight. Preferably, the same plastic is used for both housing parts, such that the powder inhaler can be readily recycled after use.

Provision can also be made that the plastic used to produce the housing parts is biodegradable, if it is not intended for recycling. Biodegradable plastics include various polyesters, polyester amides, polyurethanes and polyvinyl alcohol. Polylactic acid can also be made biodegradable by suitable mixture of the starting materials, the different isomers of the lactic acid, if appropriate as a copolymer.

For protection against counterfeits, the plastic can also contain a marker which can be verified on the finished powder inhaler. Moreover, in order to meet higher hygiene requirements, the plastic used can be an antiseptic and/or antimicrobial plastic. As an alternative thereto, an antimicrobial coating can be provided at least along the air channel.

Further embodiments, and some of the advantages associated with these and with further embodiments, will be better and more clearly understandable from the following detailed description given with reference to the accompanying figures. Objects or parts thereof which are substantially identical or similar can be provided with the same reference signs. The figures are merely a schematic representation of one embodiment of the invention.

In the figures:

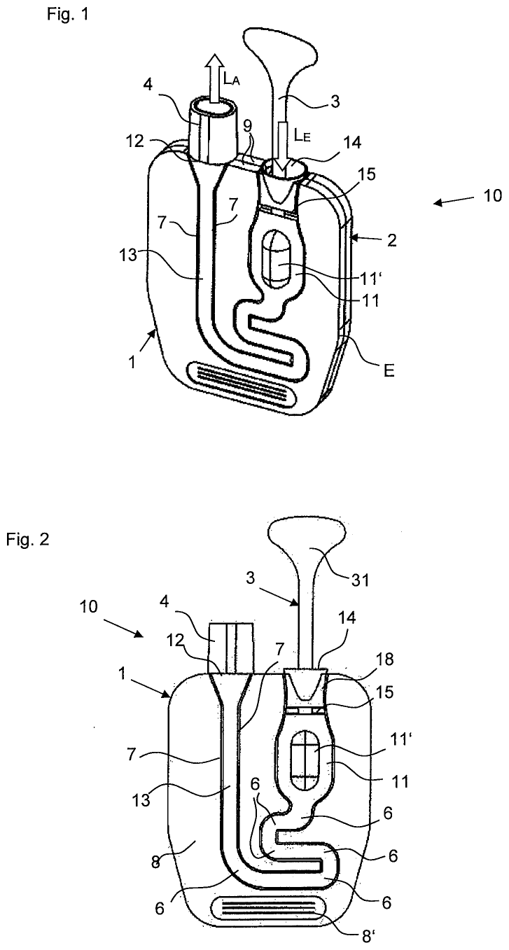

FIG. 1 shows a perspective view of an embodiment of a single-dose powder inhaler with opposite directions of air flow through air inlet opening and outlet opening, in a plane perpendicular to the connecting plane,

FIG. 2 shows a front view of the single-dose powder inhaler from FIG. 1,

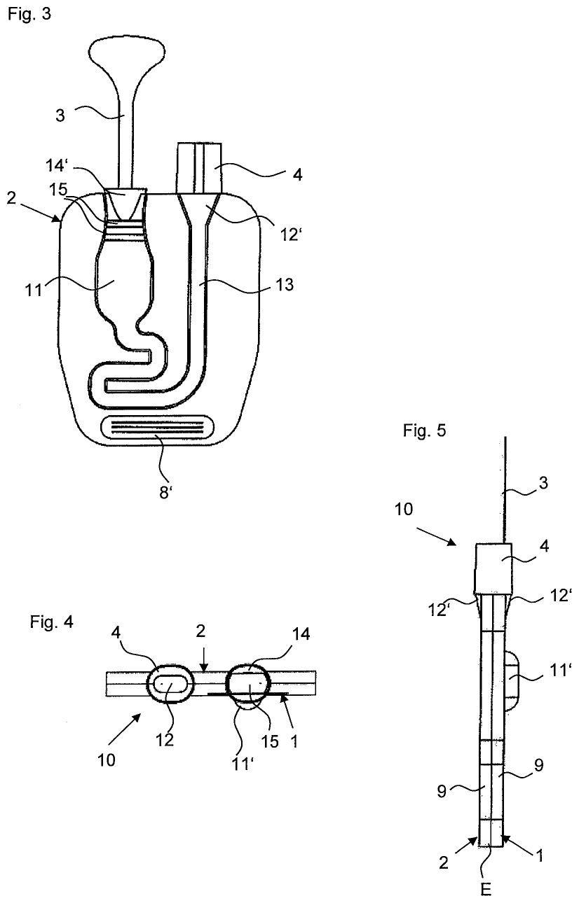

FIG. 3 shows a rear view of the single-dose powder inhaler from FIG. 1,

FIG. 4 shows a plan view of the single-dose powder inhaler from FIG. 1,

FIG. 5 shows a side view of the single-dose powder inhaler from FIG. 1,

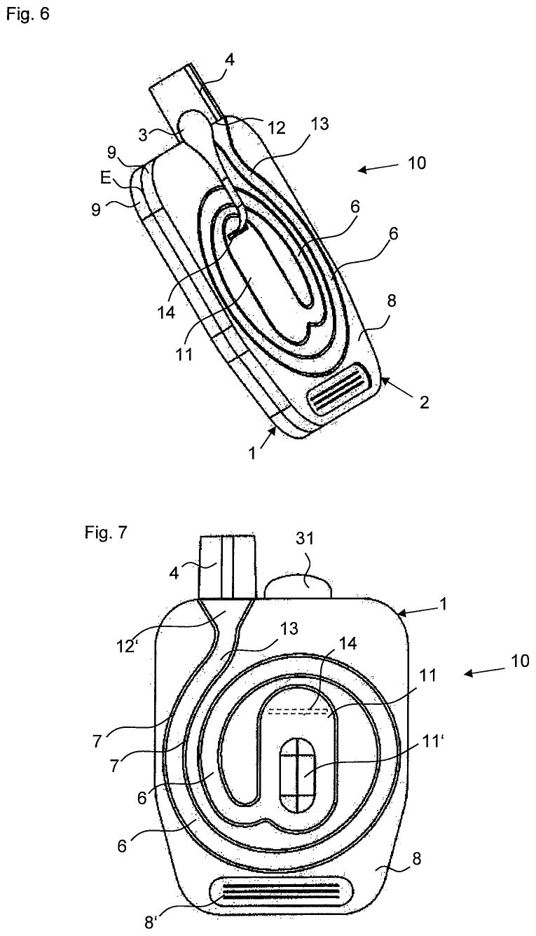

FIG. 6 shows a perspective view of a further embodiment of a single-dose powder inhaler with directions of air flow, offset by 90°, through air inlet opening and outlet opening in different planes,

FIG. 7 shows a front view of the single-dose powder inhaler from FIG. 6,

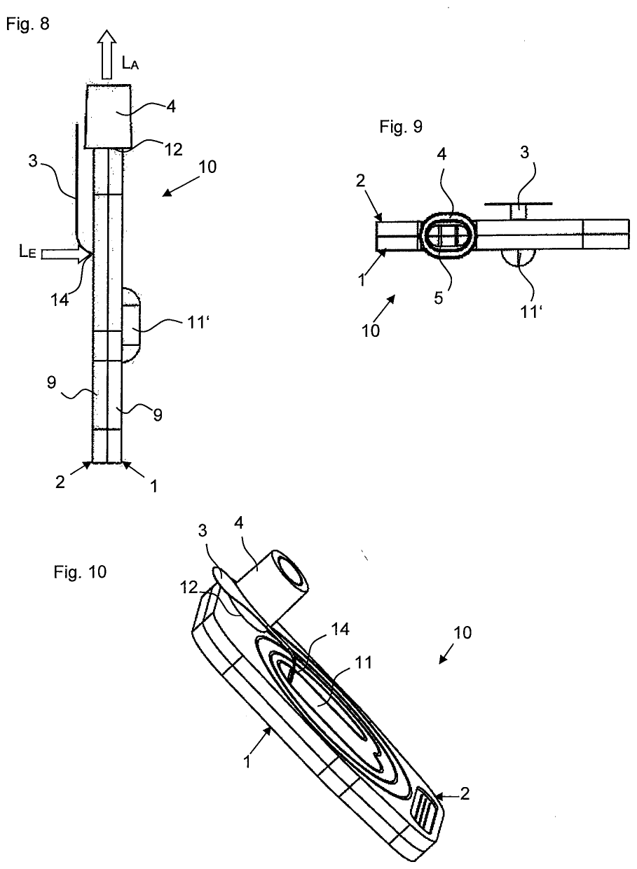

FIG. 8 shows a side view of the single-dose powder inhaler from FIG. 6,

FIG. 9 shows a plan view of the single-dose powder inhaler from FIG. 6,

FIG. 10 shows a perspective view of a further embodiment of a single-dose powder inhaler with opposite directions of air flow through air inlet opening and outlet opening, in a plane parallel to the connecting plane,

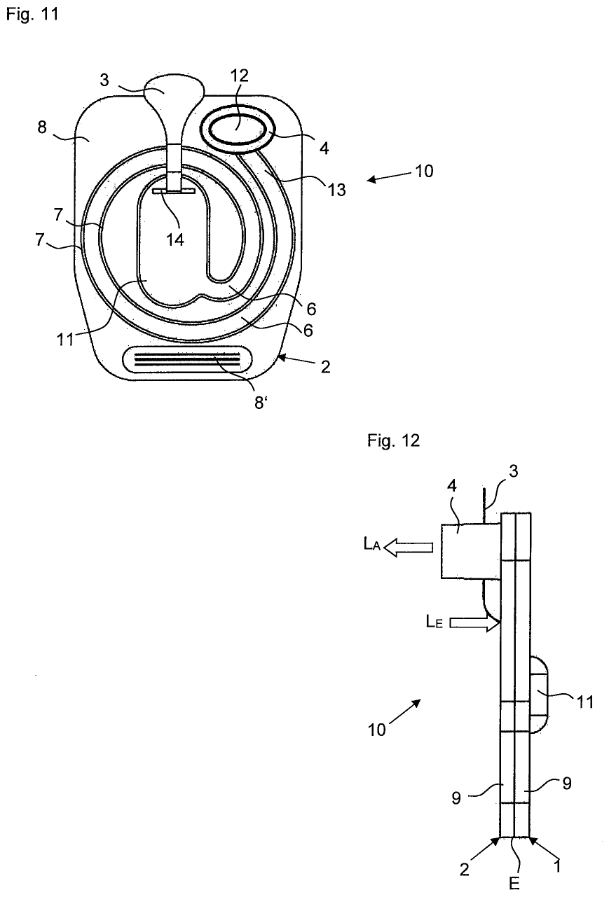

FIG. 11 shows a rear view of the single-dose powder inhaler from FIG. 10,

FIG. 12 shows a side view of the single-dose powder inhaler from FIG. 10,

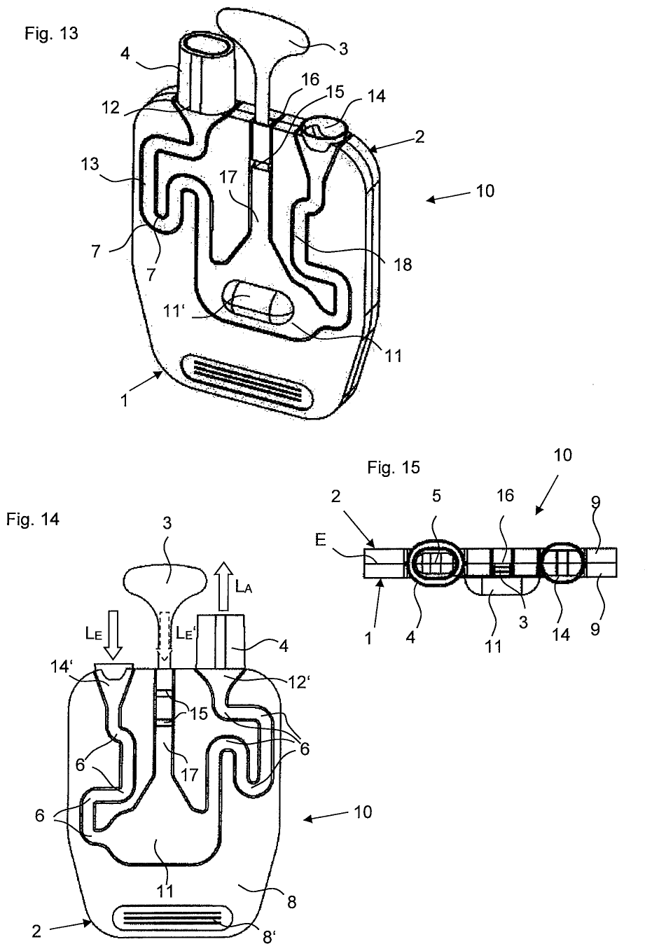

FIG. 13 shows a perspective view of yet a further embodiment of a single-dose powder inhaler with opposite directions of air flow through air inlet opening and outlet opening, in a plane parallel to the connecting plane,

FIG. 14 shows a rear view of the single-dose powder inhaler from FIG. 13,

FIG. 15 shows a side view of the single-dose powder inhaler from FIG. 13,

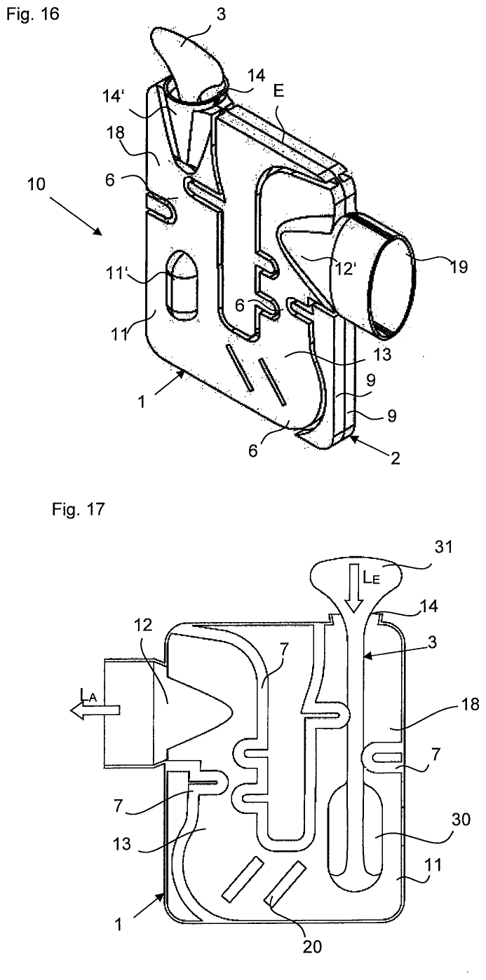

FIG. 16 shows a perspective view of an embodiment of a single-dose powder inhaler not according to the invention, with directions of air flow offset by 90° through air inlet opening and outlet opening,

FIG. 17 shows an inner view of a housing part of the single-dose powder inhaler from FIG. 16,

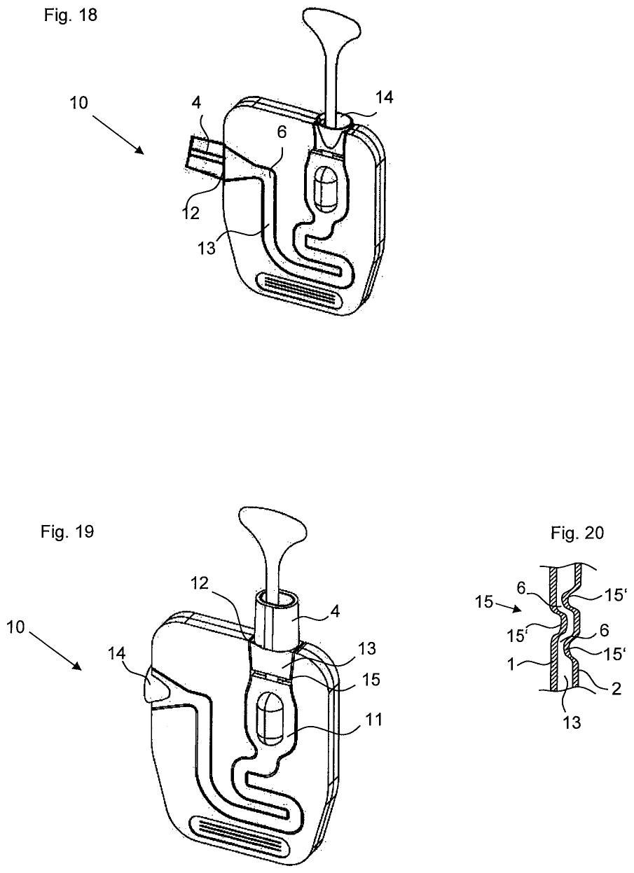

FIG. 18 shows a perspective view of a further embodiment of a single-dose powder inhaler, with directions of air flow offset by 90°,

FIG. 19 shows a perspective view of a further embodiment of a single-dose powder inhaler, with directions of air flow offset by 90° and with air inlet opening and outlet opening swapped around,

FIG. 20 shows a detailed sectional side view through a powder-retaining structure.

The device according to the invention concerns a single-dose powder inhaler which is simple and cost-effective to produce, whose housing consists of only two housing parts and which is optimized for nasal application. The single-dose powder inhaler according to the invention can be embodied in the manner of a visibility or blister pack, wherein one of the housing parts or both of them can be transparent and thus make it possible to see into the powder inhaler, in particular into the medicament chamber. It is thus possible to check the content or the complete emptying of the content after use.

As is shown in a number of illustrative embodiments in the figures, the housing of a single-dose powder inhaler 10 according to the invention is flat and of small dimensions; the inhaler is so small that it can be imagined as being about the size of a stack of 5 check cards. It can thus be easily carried around in a handbag or even in a coat pocket. The housing consists of two planar housing parts 1, 2 which are connected to each other along a connecting plane E. A medicament chamber 11 with at least one powder-receiving hollow 11′ for a dose of pulverulent medicament is formed in the housing parts 1, 2. In the examples shown in the figures, a single powder-receiving hollow 11′ is formed in a housing part 1. However, in contrast to what is shown, it is also possible to provide more than one powder-receiving hollow for partial doses or for components that are to be mixed together only at the time of use. A second or further powder-receiving hollow can be formed in the same housing part 1 or in the other housing part 2.

Moreover, an air inlet opening 14 and an outlet opening 12, and also an air channel delimited by a wall 7 on both sides, are formed in the housing parts 1, 2, which air channel connects the air inlet opening 14 to the medicament chamber 11 and connects the latter to the outlet opening 12. The arrangement of the air inlet opening 14 and of the outlet opening 12 in relation to each other is here chosen in such a way that an air inlet flow direction L_{E}, which is defined by the arrangement of the air inlet opening 14, does not correspond to the air outlet flow direction L_{A}, which is defined by the arrangement of the outlet opening 12, such that, in the case of use when the user arranges the outlet opening 12 facing upward at the nostrils, the air inlet opening 14 does not face downward, and therefore no powder can fall out of the air inlet opening 14.

Moreover, the single-dose powder inhaler 10 according to the invention has an outlet channel portion 13, which extends by more than one winding 6 from the medicament chamber 11 as far as the outlet opening 12, such that the air flow guided through the outlet channel portion 13 is subjected to several changes of direction, and a turbulent air flow is thus generated by which the powder entrained in the medicament chamber 11 is transported in a uniformly distributed manner. Moreover, for the nasal application, no deagglomerating structures are required or desirable in the outlet channel if the powder particles are intended to be deposited in the nasal mucosa and are therefore not intended to be comminuted any further.

The illustrative embodiments concern a number of variants as regards the arrangement of air inlet opening 14 and outlet opening 12 and also as regards the configuration of the sinuous outlet channel portion 13.

FIGS. 1 to 5 show various views of a first illustrative embodiment of a single-dose powder inhaler 10 according to the invention, in which the air inlet opening 14 and the outlet opening 12 are arranged on the same side at the edge 9 of the housing parts 1, 2.

The housing part 1, with the powder-receiving hollow 11′ formed in the medicament chamber 11, can be seen clearly in FIGS. 1 and 2. The housing parts 1, 2, which have a shell base 8, are connected via their edges 9 in the connecting plane E, except for the air inlet opening 14 and the outlet opening 12, as can also be seen in FIGS. 4 and 5. Although the outlet opening 12 is visible only in FIG. 4, it is also indicated in the other figures in order to illustrate the position of the outlet opening 12 at the edge 9 of the housing parts 1, 2. Formed integrally on the outlet opening 12 is a nosepiece 4 which, in the examples shown, is designed to be arranged at both nostrils. It will be seen from FIG. 4 how an outlet opening 12 provides an air flow for a nostril. The outlet opening 12 there can be formed by an edge of two overlapping half-shells, such that an elastic edge with a spring action is formed which can be placed from the inside onto a nostril.

Although not shown in the figures, the nosepiece can in principle also have two courses, in order to be used by both nostrils. The air flow from the outlet channel portion 13, which has a widening 12′ toward the outlet opening 12 in order to slow down the flow, can in this case be divided into two subsidiary flows.

The figures do not show nosepiece variants which are designed to be received in one nostril or are designed with two nozzle extensions for both nostrils but still represent nosepieces within the meaning of the invention.

As is indicated in FIG. 1 by the block arrows, the air flow direction L_{E} at the air inlet opening 14 and the air flow direction L_{A} at the outlet opening 12 (supplemented by the nosepiece 4) run in opposite directions, such that the air inlet opening 14 likewise faces upward in the case of use with the nosepiece 4 at the nostrils of a user. Before use, the film element 3 which closes the powder-receiving hollow 11′ first has to be removed. The film element 3 consists of a closure tab 30 which closes the powder-receiving hollow 11′, and a pull-off tab 31 which is connected to the closure tab 30 via a tape portion (cf. FIG. 17) and protrudes from the inhaler through the air inlet opening 14. For use, a user grips the tear-off tab 31 and pulls it in order to tear the closure tab 30 from the powder-receiving hollow 11′ and to remove the film element 3 from the inhaler 10.

By breathing in through the nose at the nosepiece 4, the user then generates an air flow through the air channel which is provided in the inhaler 10 and which is composed of an inlet channel portion 18, the medicament chamber 11 and the outlet channel portion 13, which are delimited by walls 7. These inner walls 7 of the air channel are also depicted in the figures, although they do not have to be visible from the outside, for example if the inhaler is produced from opaque material.

The air flow entering through the air inlet opening 14 upon inhalation at the nosepiece passes through the inlet channel portion 18 into the medicament chamber 11 and there entrains the powder, freed after removal of the film element 3, through the outlet channel portion 13, which opens into the outlet opening 12. The outlet channel portion 13 is here designed as a meandering channel with several curves 6. It will be appreciated that a meandering channel within the meaning of the invention can also differ from the depicted course, in terms of the number, curvature, radius and direction of the curves, without departing from the scope of protection.

Toward the outside, the inlet opening 14 has a funnel-shaped widening 14′, especially in relation to the shell base 8 of the housing parts 1, 2, as can be seen from FIGS. 3 and 4. In the inlet channel portion 18 adjoining the inlet opening 14, there is a powder-retaining structure 15 which greatly reduces the risk of powder accidentally escaping upon handling of the inhaler 10, during and after the removal of the film element 3 and before arrangement at the nose. In the examples shown, the powder-retaining structure 15 consists of barriers 15′ (cf. sectional view in FIG. 20) which extend into the air channel alternately from both housing parts 1, 2, transversely with respect to the direction of flow, and thus represent a chicane-style obstacle and narrow the cross section of the inlet channel portion at this location, as can also be seen in FIG. 4. In the example shown, housing part 1 has a barrier 15′ and housing part 2 has two barriers 15′ arranged in an offset position relative thereto. Although not described explicitly here, it is readily conceivable here for a powder-retaining structure to deviate in terms of the number and profile of the barriers.

On a side of the powder inhaler 10 directed away from the outlet opening 12/nosepiece 4, a ribbed structure 8′ is provided on the surface in the shell base 8 of each of the housing parts 1, 2, which ribbed structure 8′ serves for an improved grip of the inhaler 10 when it is held by a user.

For the embodiments described in connection with the further figures, only those features that differ from the example described with reference to FIGS. 1 to 5 will be described below.

FIGS. 6 to 9 show a single-dose powder inhaler 10 according to the invention in which the air inlet opening 14 is not at the edge but is instead formed in the shape of a slit in the shell base 8 of the housing part 2. The air inlet opening 14 opens directly here into the medicament chamber 11, above the powder-receiving hollow 11′, which is formed in the other housing part 1. Here too, the film element 3, which closes the powder-receiving hollow 11′ with a closure tab (not shown here), extends out of the inhaler 10 with the tear-off tab 31 through the air inlet opening 14. Since there is no inlet channel as such in this embodiment, this inhaler 10 has no separate powder-retaining structure. The latter can be omitted here by virtue of the slit-shaped configuration of the air inlet opening 14 and the arrangement of the latter above the powder-receiving hollow 11′, since unwanted escape of powder is here also avoided by the simplified process of removal of the film element 3, in which less force needs to be applied to release and pull the film element 3 out in the tear-off direction, as a consequence of the arrangement of the powder-receiving hollow 11′ relative to the air inlet opening 14 and the associated short dimension of the tape portion that is to be pulled out of the inhaler.

It will be noted that the slit-shaped air inlet opening shown can also be differently configured; instead of a slit, another geometry can be chosen, or further openings are present in the manner of a bypass opening.

As the block arrows in FIG. 8 show, the air inlet opening 14 and the outlet opening 12 are here arranged at right angles to each other, such that the air inlet flow direction L_{E} and the air outlet flow direction L_{A} run at an angle of ca. 90° to each other. The air inlet opening 14 located in the shell base 8 lies in a plane parallel to the connecting plane E, while the edge-side outlet opening 12 is arranged perpendicularly thereto.

The outlet channel portion 13 of the powder inhaler from FIGS. 6 to 9 runs in a spiral shape with two windings 6 about the medicament chamber 11 and forms a widening 12′ in front of the outlet opening 12 at the nosepiece 4.

FIGS. 10 to 12 show a single-dose powder inhaler 10 according to the invention which differs from the powder inhaler of FIGS. 6 to 9 only in terms of the arrangement of the outlet opening 12 and of the nosepiece 4. Here, the outlet opening 12 is likewise present in the shell base 8 of the housing part 2, such that the nosepiece 4, adjoining the outlet opening 12, and also the air outlet direction L_{A} and the oppositely directed air inlet direction L_{E} extend perpendicularly with respect to the connecting plane E.

The embodiment of a single-dose powder inhaler 10 according to the invention shown in connection with FIGS. 13 to 15 again has a meandering outlet channel portion 13 and, arranged at the edge on one side, an air inlet opening 14 and outlet opening 12, such that air outlet direction L_{A} runs counter to air inlet direction L_{E}. Here, however, the inlet channel portion 18 also has several windings 6. On account of the several windings 6, it is possible to do without a further powder-retaining structure in the inlet channel portion 18 of this embodiment; however, the meandering inlet channel portion 18 also prevents the pulling-through of a film element which is provided for closing the powder-receiving hollow 11′ in the medicament chamber 11. Therefore, this powder inhaler 10 has an additional channel 17 which extends from a likewise edge-side opening 16, between the air inlet opening 14 and the outlet opening 12, in a straight line into the medicament chamber 11. The film element 3 closing the powder-receiving hollow 11′ extends through this channel 17 and protrudes with the tear-off tab out of the opening 16 which, after the film element 3 has been pulled out, acts as an additional air inlet opening during use, as is indicated by the dashed block arrow L_{E}′ in FIG. 14. On account of the rectilinear course of the tab channel 17, a powder-retaining structure 15 is arranged there in order to avoid unwanted emergence of powder. This powder-retaining structure corresponds to the barrier arrangement in the inlet channel portion, which was described in connection with the embodiment shown in FIGS. 1 to 5.

The single-dose powder inhaler 10 shown in FIGS. 16 and 17 is an embodiment not according to the invention, in which a mouthpiece 19 is integrally formed at the outlet opening 12, such that this powder inhaler 10 is provided for use by oral inhalation. For a turbulent air flow into and out of the medicament chamber 11, the inlet channel portion 18 and the outlet channel portion 13 are designed in a meandering configuration with curves 6. For better distribution of the powder particles and, if appropriate, also for comminution of powder agglomerates that have formed, baffle walls 20 are here provided in the outlet channel portion 13. The outlet opening 12 is, like the inlet opening 14, formed at the edge in the housing parts 1, 2, but at adjacent sides, such that air inlet flow direction L_{E} and air outlet flow direction L_{A} form an angle of 90°.

In FIG. 17, which shows the housing part 1 from the inside, it is also possible to see the complete film element 3, of which the closure tab 30 closes the thereby concealed powder-receiving hollow. The film element 3 can be made of aluminum, plastic or a composite film. As can be seen in FIG. 17, the tape portion of the film element 3 coming from the tear-off tab 31 extends as far as the distal side of the closure tab 30, such that, by pulling on the tear-off tab 31, the closure tab 30, which is secured to the housing part 1 by adhesive bonding or sealing, can be pulled away from the distal side and can then be removed completely from the inhaler 10. The powder dose contained in the now opened powder-receiving hollow 11′ is thereby released and is entrained in an air flow that is generated by an inhalation at the outlet opening 12.

Except for the mouthpiece, the inhalers described in connection with FIGS. 16 and 17 correspond to embodiments according to the invention. That is to say, the details described, except for the mouthpiece, are transferable to embodiments according to the invention if, instead of the mouthpiece, a nosepiece is integrally formed at the outlet opening.

The outlet opening 12 and the inlet opening 14 of the single-dose powder inhaler 10 shown in FIG. 18 are arranged correspondingly, although this single-dose powder inhaler 10 has a nosepiece 4 and is thus designed in the preferred embodiment for nasal application. This powder inhaler 10 corresponds to the one shown in FIGS. 1 to 5, except for the different arrangement of outlet opening 12 and inlet opening 14 and for the resulting additional curve 6 in the outlet channel portion 13.

FIG. 19 shows a single-dose powder inhaler 10 in which, compared to the one shown in FIG. 18, the air inlet opening 14 and the outlet opening 12 are changed around. In contrast to the other embodiments, the powder-retaining structure 15, which is present here in the outlet channel portion 13, has barriers 15′ extending alternatingly into the channel and thus providing the windings 6 (cf. FIG. 20) that ensure the required several changes of direction of the air flow guided through the outlet channel portion 13.

In the embodiments described as examples in connection with the figures, the air inlet opening 14 and the outlet opening 12 are in each case arranged in relation to each other in such a way that the air inlet flow direction L_{E} and the air outlet flow direction L_{A} run at an angle of 90° to each other or at 180° (oppositely directed). However, it will be appreciated that arrangements can also be provided which differ from this and which lie within the scope of protection of the invention as long as air inlet opening and outlet opening do not lie in the straight-ahead direction but are instead moreover preferably arranged such that the air inlet flow direction L_{E} and the air outlet flow direction L_{A} span an angle of at least 90°, in order to effectively prevent escape of the powder in the position of use of the inhaler with the outlet opening directed substantially perpendicularly upward.

Particularly if the inhaler housing does not have a substantially rectangular base (or a shape composed of rounded rectangle and trapezium) as in the examples shown, but instead has, for example, an oval or regular hexagonal shape, the air inlet opening and outlet opening can deviate from the illustrated arrangements with flow directions offset by 90° or 180°.

Moreover, the illustrative embodiments shown in the figures show housing parts with a shell base 8, wherein an outer surface of the housing is substantially plane except for the powder-receiving hollow and the widenings to inlet opening and outlet opening. The figures do not show embodiments of a single-dose powder inhaler according to the invention in which the housing parts are based on a plate base. However, as a person skilled in the art can readily imagine, the corresponding structures in the housing parts are then formed in plate elements instead of in shell elements, such that the structures rise from the flat plate base. While the housing parts 1, 2 on shell base 8 are connected to each other at their edges 9 and at the wall 7 delimiting the air channel and the medicament chamber 11, housing parts on a plate base can be connected to each other in a planar manner in the connecting plane outside the shaped regions.

The housing parts of a single-dose powder inhaler according to the invention can be welded to each other by heat, but adhesive bonding, clamping connections (wherein the edge of one housing part is bent around the edge of the other housing part), latching connections or stitched connections also come into question.

The powder inhaler according to the invention is provided for single use, and therefore the inhaler housing cannot be used again after the powder-receiving hollow has been opened and the dose of powder has been inhaled, and this also serves to protect against misuse. In addition to the good recyclability or the biodegradability, the increased volume of packaging waste, which is considered a drawback from the point of view of environmental protection, is also offset, however, by the reduced use of multidose inhalers, the disposal of which is more complicated since residual quantities of the pulverulent medicament are usually contained therein.

The single-dose powder inhaler according to the invention permits economical use for various indications, for instance when only a few uses are necessary, or when the inhaler is used only when required. The powder inhaler according to the invention is hygienic, and undesired influences such as high air humidity or soiling on account of misuse are largely ruled out. By virtue of the cost-effective production, the powder inhaler according to the invention is also suitable for use in the Third World and in crisis regions.

In fact, the powder inhaler according to the invention is so favorable in terms of its production that a patient-specific medicament blister pack can be realized in an economically justifiable manner. The composition and dosing of the pulverulent medicament (or plurality of medicaments) can thus be filled into the powder inhaler according to the invention in a specific manner tailored to each patient.

The plastic used to produce a powder inhaler according to the invention, designed as a disposable item, can preferably be a biodegradable plastic. Moreover, the plastic can be either a plastic with antiseptic or antibacterial and/or antimicrobial properties or can be provided at least partially with an antiseptic or antibacterial and/or antimicrobial coating. Thus, in particular the air inlet channel, medicament chamber and air outlet channel can be coated in order that no germs are inhaled when using the inhalation device. However, on account of the easier application, it is also possible for the entire inhalation device to be coated. An example of such a coating is Perlazid® from Rilit-Lackfabrik GmbH, Endingen, Germany.

A powder inhaler according to the invention can also be marketed for example in an outer packaging which ensures that the powder inhaler is clean or sterile and thus immediately ready for use. Alternatively, it may also be conceivable for a cover element to be provided at the air inlet opening and the outlet opening, with which cover element the openings can be closed, for example, in the manner of a yogurt pot lid.

Finally, the plastic used to produce a powder inhaler according to the invention can have a marker for the identification thereof, in order for it to be possible to identify counterfeits that do not contain the marker.

### LIST OF REFERENCE CHARACTERS

1 housing part with powder capsule
2 housing part
3 film element
30 closure tab
31 tear-off tab
4 nosepiece
6 winding
7 air channel wall
8 shell base
8′ ribbing
9 edge
10 single-dose powder inhaler
11 medicament chamber
11′ powder-receiving hollow
12 outlet opening
12′ widening
13 outlet channel
14 air inlet opening
14′ widening
15 powder-retaining structure
15′ barrier
16 tab opening
17 tab channel
18 inlet channel
19 mouthpiece
20 baffle wall
E connecting plane
L_{A} air outlet flow direction
L_{E} air inlet flow direction

What is claimed is:

1.-9. (canceled)

10. A single-dose powder inhaler (10) for nasal application, the single-dose powder inhaler comprising:
a flat inhaler housing comprised of two planar housing parts (1, 2) connected to each other along a connecting plane (E) and comprising, integrally formed therein as shaped regions, a medicament chamber (11) with a powder-receiving hollow (11′), an air inlet opening (14), an outlet opening (12), and an air channel delimited by a wall (7) and extending from the air inlet opening (14) via the medicament chamber (11) to the outlet opening (12);
a nosepiece (4), configured to be received in a nostril or in both nostrils, integrally formed at the outlet opening (12);
wherein the air inlet opening (14) is not arranged on a side of the inhaler housing facing away from the outlet opening (12);
wherein the air channel is configured to cause an air flow flowing from the air inlet opening (14) to the outlet opening (12) to undergo a directional change;
wherein the air channel comprises an outlet portion (13) arranged between the medicament chamber (11) and the outlet opening (12), wherein the outlet portion (13) comprises first windings and no deagglomerating structure.

11. The single-dose powder inhaler (10) as claimed in claim 10, wherein the air inlet opening (14) and the outlet opening (12) are arranged in a neighboring position next to each other or are arranged offset from each other by 90° or by an angle between 90° and the neighboring position.

12. The single-dose powder inhaler (10) as claimed in claim 10, wherein the air inlet opening (14) and the outlet opening (12) are arranged next to each other perpendicularly to or parallel to the connecting plane (E) of the housing parts (1, 2).

13. The single-dose powder inhaler (10) as claimed in claim 10, wherein one of the air inlet opening (14) and the outlet opening (12) is arranged parallel to the connecting plane (E) of the housing parts (1, 2) and the respective other one of the air inlet opening (14) and the outlet opening (12) is arranged perpendicularly to the connecting plane (E) of the housing parts (1, 2).

14. The single-dose powder inhaler (10) as claimed in claim 10, wherein the outlet portion (13) of the air channel is a meandering channel and the first windings include at least three curves, or the outlet portion (13) of the air channel comprises a spiral-shaped channel with at least two of the first windings.

15. The single-dose powder inhaler (10) as claimed in claim 10, further comprising at least one tear-off film element (3) comprising a closure tab (30) closing the powder-receiving hollow (11′) in the medicament chamber (11) and further comprising a tear-off tab (31) which extends away from the closure tab (30) out of the medicament chamber (11) and out of the air inlet opening (14) by passing through an inlet portion (18) of the air channel that is disposed between the air inlet opening (14) and the medicament chamber (11).

16. The single-dose powder inhaler (10) as claimed in claim 15, wherein the inlet portion (18) of the air channel comprises a powder-retaining structure (15), wherein the powder-retaining structure (15) narrows a channel cross section of the air channel and is at least one barrier (15′) that extends into the air channel transversely to a flow direction of the air flow.

17. The single-dose powder inhaler (10) as claimed in claim 15, wherein the inlet portion (18) of the air channel comprises second windings such that the air flow guided through the inlet portion (18) changes the flow direction several times.

18. The single-dose powder inhaler (10) as claimed in claim 10, further comprising at least one tear-off film element (3) comprising a closure tab (30) closing the powder-receiving hollow (11′) in the medicament chamber (11) and further comprising a tear-off tab (31) which extends away from the closure tab (30) out of the medicament chamber (11) and out of a tab opening (16) by passing through a tab channel (17) which extends between the tab opening (16) and the medicament chamber (11).

19. The single-dose powder inhaler (10) as claimed in claim 18, wherein the tab channel (17) comprises a powder-retaining structure (15), wherein the powder-retaining structure (15) narrows a channel cross section of the tab channel (17) and is at least one barrier (15′) that extends into the tab channel (17) transversely to a flow direction of the air flow.

20. The single-dose powder inhaler (10) as claimed in claim 10, wherein the medicament chamber (11) comprises two or more of said powder-receiving hollow (11′), wherein the two or more of said powder-receiving hollow (11′) each contain a partial dose of a pulverulent medicament.

21. The single-dose powder inhaler (10) as claimed in claim 20, further comprising a tear-off film element (3) for each one of the two or more of said powder-receiving hollow (11′), wherein the tear-off film element comprises a closure tab (30) closing the respective powder-receiving hollow (11′) and further comprising a tear-off tab (31) which extends away from the closure tab (30) out of the medicament chamber (11) to an exterior of the flat inhaler housing.

22. The single-dose powder inhaler (10) as claimed in claim 20, further comprising a tear-off film element (3) for the two or more of said powder-receiving hollow (11′), wherein the tear-off film element comprises a closure tab (30) closing the two or more of said powder-receiving hollow (11′) and further comprising a tear-off tab (31) which extends away from the closure tab (30) out of the medicament chamber (11) to an exterior of the flat inhaler housing.

23. The single-dose powder inhaler (10) as claimed in claim 10, wherein the housing parts (1, 2) are plate elements connected to each other in a planar manner in the connecting plane (E) outside the shaped regions.

24. The single-dose powder inhaler (10) as claimed in claim 10, wherein the housing parts (1, 2) are shell elements with circumferential edges (9) connected to each other in the connecting plane (E) at least at the circumferential edges (9).

25. The single-dose powder inhaler as claimed in claim 10, wherein the housing parts (1, 2) are injection-molded plastic parts comprised of a plastic material, wherein the plastic material is biodegradable and/or contains a marker and/or is antiseptic and/or antimicrobial.

26. The single-dose powder inhaler as claimed in claim 10, wherein the housing parts (1, 2) are shaped or thermoformed from a pharmaceutically acceptable single-type plastic film, wherein a plastic material of the plastic film is biodegradable and/or contains a marker and/or is antiseptic and/or antimicrobial.

27. The single-dose powder inhaler as claimed in claim 10, wherein the housing parts (1, 2) comprise an antiseptic and/or antimicrobial coating at least along the air channel.

28. The single-dose powder inhaler as claimed in claim 10, wherein at least one of the housing parts (1, 2) is comprised of a transparent plastic material.
